(19) United States
(12) Patent Application Publication
(10) Pub. No.: US 2001/0029266 A1
(43) Pub. Date: Oct. 11, 2001

(54) Composition containing a pentacyclic triterpenic acid, method

(75) Inventors: Laurence Leroy, Rambouillet (FR); Veronique Burnier, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL

(21) Appl. No.: 09/731,865

(22) Filed: Dec. 8, 2000

(30) Foreign Application Priority Data
Dec. 10, 1999 (FR) 9915612

(51) Int. Cl. A61K 31/19

(52) U.S. Cl. 514/557

(57) ABSTRACT

Composition for containing at least 0.02% of at least one pentacyclic triterpenic acid and at least one emulsifier, liquid at 25° C., which is an optionally oxyalkylenated fatty acid monoester, having an HLB greater than or equal to 12 method of preparation.

### BACKGROUND OF THE INVENTION

1. Field of the Invention

The present invention relates to a method of solubilizing a pentacyclic triterpenic acid. It also relates to novel compositions comprising, preferably in a physiologically acceptable medium, at least one pentacyclic triterpenic acid and at least one emulsifier, liquid at 25° C., which is a fatty acid monoester having an HLB greater than or equal to 12. It also relates to the use of the abovementioned compositions in a cosmetic preparation, or for the manufacture of a preparation intended in particular for the care of the skin, the mucous membranes and/or the keratinous fibres.

2. Background

The pentacyclic triterpenic acids such as ursolic acid and oleanolic acid are present in plants such as rosemary. They are frequently used in pharmaceutical compositions for their numerous therapeutic properties, and for example for their anti-inflammatory, hepatoprotective, diuretic, analgesic, antimicrobial, enzymatic activity-inhibiting and antitumour properties. They are thus described in the document JP-2017121 for the prevention of skin cancer. In the cosmetic field, ursolic acid is described for example as a constituent of an antiperspirant composition in the document FR-A-2,541,895 and as inhibitor of the activity of tyrosinase in the document JP-58/57307.

The pentacyclic triterpenic acids are therefore of great interest given their biological effects on the skin. However, the formulation of these compounds is problematic since these compounds exist in the form of crystals which are insoluble or sparingly soluble in water and in the oils or solvents traditionally used in the cosmetic and dermatological fields. This low solubility of the pentacyclic triterpenic acids hampers the efficacy of the compositions containing them and their perception by the users.

Thus, according to the document by Jin et al., Arch. Pharm. Res., 1997, Vol. 20, No 3, p. 275 to 279,surfactants and most solvents (such as polyethylene glycols) are not very appropriate or allow only limited solubilization of ursolic acid.

It has however been proposed to solubilize pentacyclic triterpenic acids in aqueous solutions having a high concentration of ionic surfactants or of nonionic surfactants consisting of ethers of fatty alcohols and of polyethylene glycol. The use of such quantities of this type of surfactants is however not appropriate for the preparation of cosmetic compositions since they cause phenomena including irritation, overheating and redness which are unacceptable for a cosmetic composition which should be free of any side effects likely to deter the user from its use.

Another solvent proposed in the above publication for solubilizing pentacyclic triterpenic acids is tetramethylurea. Apart from the fact that its use is limited in cosmetics for problems of skin tolerance, tetramethylurea constitutes a good solubilizing agent for ursolic acid only in the case where the quantity of water present in the composition is less than 30% or even less than 20%. However, cosmetic compositions in the form of nonfatty lotion or fluids, or even of creams, frequently contain more than 50% by weight of water. Because of these two reasons, tetramethylurea is not suitable for cosmetic use.

The document JP-2,017,121 discloses examples of cosmetic compositions containing ursolic acid and/or oleanolic acid. In Example 1, a cream is prepared by mixing an aqueous-alcoholic solution of neutralized ursolic acid, heated to 80° C., with a mixture of molten fatty substances heated to the same temperature. The ursolic acid represents 0.01% by weight of the total composition. The mixture of fatty substances contains a polyoxyethylenated sorbitan monostearate which is very likely either solid (of the Tween 61® type) or semisolid (of the Tween 60® type). This type of emulsifier does not make it possible to solubilize a sufficient quantity of ursolic acid without loss of stability of the composition, in particular when the ursolic acid is present in the free acid form.

### OBJECT OF THE INVENTION

There is still therefore the need to be able to introduce a quantity of at least 0.02% by weight of pentacyclic triterpenic acid into a composition, in particular a cosmetic composition, which is stable and substantially nonirritant, in which the triterpenic acid will not tend to recrystallize during the period of storage and use of the composition.

This object and others will become apparent from the following Summary and Detailed Description of the Invention.

### SUMMARY OF THE INVENTION

The inventors have discovered, unexpectedly, that the above object is satisfied, according to the invention, by the use of certain fatty acid monoesters as emulsifier.

### DETAILED DESCRIPTION OF THE INVENTION

One subject of the present invention is therefore a composition preferably useful for topical application comprising, preferably in a physiologically acceptable medium, at least 0.02% of at least one pentacyclic triterpenic acid and at least one emulsifier, the at least one emulsifier being liquid at 25° C., which at least one emulsifier is an optionally oxyalkylenated fatty acid monoester, having an HLB greater than or equal to 12.

The pentacyclic triterpenic acid is preferably chosen from ursolic acid and oleanolic acid. It is preferably essentially in free acid form, that is to say in an amount of at least 50% by weight. Needless to say, in invention compositions, more than one pentacyclic triterpenic acid and more than one monoester may be present.

The term fatty acid monoester is understood to mean a monoester formed from a fatty acid having from 12 to 22 carbon atoms and either from a polyol, optionally oxyalkylenated, or from a polyalkylene glycol, the ester obtained being sufficiently hydrophilic for its HLB to be greater than or equal to 12, preferably between 12 and 17. The fatty acid monoesters formed from a fatty acid comprising from 12 to 22 carbon atoms and from polyglycerol, and the fatty acid monoesters formed from a fatty acid comprising from 12 to 22 carbon atoms and from polyoxyethylenated sorbitan, are preferred in particular.

Oxyalkylenated refers to an ester formed from fatty acid and polyol which has been condensed with moles of alkylene oxide, such as ethylene oxide. The OH groups of the polyol are condensed with the alkylene oxide.

Another subject of the invention is a method of solubilizing a pentacyclic triterpenic acid, comprising mixing at room temperature one or more such acids with at least one optionally oxyalkylenated fatty acid monoester, liquid at 25° C. and having an HLB greater than or equal to 12.

The fatty acid monoester is present, in the compositions according to the invention, in a quantity sufficient to allow the solubilization of the pentacyclic triterpenic acid and to obtain a stable composition, without recrystallization of the triterpene. One of ordinary skill is capable of determining this amount. The expression “stable” is understood to mean that the pentacyclic triterpenic acid does not recrystallize when the composition is kept for at least four weeks at room temperature. It is also understood to mean the absence of phase separation, creaming and sedimentation.

Of course, the exact quantity of fatty acid monoester will depend on the quantity of pentacyclic triterpenic acid. As a guide, the pentacyclic triterpenic acid preferably represents from 0.05 to 5%, such as 0.1, 0.5, 1, 1.5, 2, 2.5, 3, 3.5, 4 and 4.5, or better from 0.4 to 2%, or even better still about 1%, of the total weight of the composition. Under these conditions, the fatty acid monoester advantageously represents from 0.05 to 10% such as 0.1, 0.5, 1, 2, 3, 4, 5, 6, 7, 8, 9, and, preferably, from 1 to 5%, of the total weight of the composition.

As fatty acid monoesters, useful examples include:

monoesters of a fatty acid and of polyglycerol, such as hexaglyceryl monolaurate, decaglyceryl monoisostearate, decaglyceryl monolaurate, decaglyceryl monolinoleate, decaglyceryl monomyristate and decaglyceryl monooleate;

monoesters of a fatty acid and of sorbitan/sorbitol which are polyoxyethylenated, such as polyoxyethylenated sorbitan monostearate, polyoxyethylenated (20 EO) sorbitan monolaurate, polyoxyethylenated (4 EO) sorbitan monolaurate, polyoxyethylenated (20 EO) sorbitan monooleate, polyoxyethylenated (40 EO) sorbitol tetraoleate and polyoxyethylenated (60 EO) sorbitol tetraoleate; and

monoesters of a fatty acid and of polyalkylene glycol, such as polyethylene glycol monooleate (600 EO), polyethylene glycol monolaurate (400 EO), polyoxyethylenated castor oil (40 EO), polyoxyethylenated castor oil (60 EO) and polyoxyethylenated hydrogenated castor oil (40 EO).

These compounds have the advantage of avoiding the recrystallization of the pentacyclic triterpenic acids and of allowing their incorporation into the aqueous phase of cosmetic or dermatological compositions, without causing skin irritation.

In this regard, it has been discovered that pentacyclic triterpenic acid can be incorporated, without recrystallization, into compositions containing large quantities of aqueous phase. Thus, the composition according to the invention may for example contain from 30 to 95% by weight of water, advantageously from 50 to 80% by weight of water.

This aqueous phase may contain, in addition, in particular one or more alcohols and/or polyols such as ethanol, glycerin, butylene glycol, isoprene glycol, propylene glycol, sorbitol, in concentrations preferably ranging from 1 to 20% by weight, relative to the total weight of the composition.

The composition according to the invention may be provided in any galenic form appropriate for topical use and in particular in the form of an aqueous solution, an aqueous gel, a dispersion, an oil-in-water (O/W) or water-in-oil (W/O) emulsion, a triple emulsion (W/O/W or O/W/O). They may also be in a vectorized form, such as for example in the form of nanocapsules, liposomes, nanoemulsions or oleosomes.

According to a preferred embodiment of the invention, the composition is a W/O or O/W emulsion.

The composition may be used for topical application and appropriately preferably comprises a physiologically acceptable medium. The expression physiologically acceptable medium is understood to mean a medium compatible with the skin, the mucous membranes (including the lips) and the superficial body growths (nails, hair and eyelashes).

When the composition is an emulsion, the latter conventionally contains at least one oil, an aqueous phase and optionally an appropriate emulsifier, in addition to the fatty acid monoester according to the invention.

The nature of the oily phase entering into the composition according to the invention is not critical and it may thus use all the fatty substances and in particular the oils conventionally used in the cosmetic and dermatological fields.

Among the oils which may be used in the emulsion of the invention, examples in particular include vegetable oils, mineral oils, synthetic oils, silicone oils and fluorinated oils. Other fatty substances which may be present in the oily phase may be for example fatty acids, fatty alcohols and waxes.

The oily phase of the emulsion may represent from 1 to 70%, and even better from 5 to 40% of the total weight of the emulsion.

The emulsions may contain at least one emulsifying agent, other than the fatty acid monoester according to the invention, which makes it possible to stabilize the oil/water interface. This additional emulsifier may be chosen from anionic, cationic or nonionic emulsifiers, used alone or in the form of a mixture. These additional emulsifiers are appropriately chosen according to the emulsion to be obtained (W/O or O/W).

Depending on the fluidity which it is desired to confer on the composition, it is also possible to add thereto one or more gelling agents which may represent, for example, from 0.1 to 10%, and preferably from 0.1 to 5%, or even better from 0.1 to 3% of the total weight of the composition.

In addition, in a known manner, the composition of the invention may contain adjuvants customarily used in the cosmetic and dermatological fields, such as hydrophilic or lipophilic active agents, preservatives, antioxidants, perfumes, fillers, colouring matter (pigments or colorants) and sunscreens. These adjuvants are used in the proportions customarily used in the cosmetic and dermatological fields, and for example in an amount of 0.01 to 20% of the total weight of the composition. Depending on their nature, they are introduced into the aqueous phase or into the oily phase of the composition, or alternatively into vesicles. The nature and the quantity of these adjuvants should however preferably be such that they do not modify the properties desired for the composition according to the invention.

The composition according to the invention is advantageously obtained by mixing, at room temperature, generally, but optionally, in the presence of water, the pentacyclic triterpenic acid with the fatty acid monoester, liquid at 25° C. and having an HLB greater than or equal to 12.

The invention method preferably comprises the additional two steps of homogenizing the mixture at room temperature and then introducing it into an O/W or W/O emulsion, also at room temperature.

The compositions according to the invention find application for example in a large number of cosmetic and dermatological treatments of the skin, the mucous membranes and/or superficial body growths, in particular for the protection of, the care of, the cleansing of and/or the application of make-up to the skin and/or the mucous membranes, for the protection of and/or the care of the hair and/or for the dermatological treatment of the skin, of the hair and/or of the mucous membranes.

To this end, the composition according to the invention may be used in care or cleansing products for the face or the body, in the form of lotions, creams or milks, in make-up products by incorporation of fillers, pigments or colorants, or alternatively in antisun products, by incorporation of sunscreens.

Thus, one subject of the invention is a cosmetic method for the prevention or treatment of acne, of photoageing and/or of sensitive skins, comprising the topical application of the compositions described above.

Another subject of the invention is the use of the compositions described above for the manufacture of a preparation intended for the prevention or treatment of acne and/or of solar erythema and/or of sensitive skins.

Other characteristics and advantages of the invention will emerge more clearly from the following exemplary embodiments given by way of illustration and without limitation. In these examples, unless otherwise stated, the quantities are indicated in percent by weight.

### EXAMPLES

### Example 1

### O/W Emulsion

| | |
|---|---|
| Phase A | |
| Polyoxyethylene glycol stearate (100 EO) and glyceryl | 2.5% |
| stearate | |
| Polyethylene glycol monostearate (50 EO) | 2.5% |
| Stearyl alcohol | 1% |
| Cetyl alcohol | 1% |
| Hydrogenated polyisobutene | 20% |
| Phase B | |
| Carbomer (carboxylvinyl polymer) | 0.3% |
| Triethanolamine | 0.3% |
| Water | 10% |
| Phase C | |
| Sorbitan monooleate | 4% |
| Ursolic acid (supplied by NIKKOL) | 1.6% |
| Water | qs 34.4% |
| Phase D | |
| Preservatives | qs |
| Water | qs 100% |

The above composition was prepared in the following manner. An oil-in-water emulsion was first of all conventionally prepared by introducing, with stirring, at 70-75° C., phase A into phase D. Phase B was prepared at 70-75° C. and then added at 40-50° C., with stirring, to the emulsion obtained above. Phase C was prepared by mixing its constituents at room temperature and homogenizing for 48 hours, and then it was added, with stirring, to the mixture of phases A, B and D previously cooled to room temperature.

No recrystallization of ursolic acid was observed after one month at room temperature and at 45° C.

### Example 2

### O/W Emulsion

The following composition was prepared in the same manner as in Example 1.

| | |
|---|---|
| Phase A | |
| Polyoxyethylene glycol stearate (100 EO) and glyceryl | 2.5% |
| stearate | |
| Polyethylene glycol monostearate (50 EO) | 2.5% |
| Stearyl alcohol | 1% |
| Cetyl alcohol | 1% |
| Hydrogenated polyisobutene | 20% |
| Phase B | |
| Carbomer (carboxylvinyl polymer) | 0.3% |
| Triethanolamine | 0.3% |
| Water | 10% |
| Phase C | |
| Polyglycerolated monooleate | 1% |
| Ursolic acid (supplied by NIKKOL) | 0.4% |
| Water | qs 8.6% |
| Phase D | |
| Preservatives | qs |
| Water | qs 100% |

No recrystallization of ursolic acid was observed after one month at room temperature and at 45° C.

### Example 3 (Comparative)

The following composition was prepared in the same manner as in Example 1.

| | |
|---|---|
| Phase A | |
| Polyoxyethylene glycol stearate (100 EO) and glyceryl | 2.5% |
| stearate | |
| Polyethylene glycol monostearate (50 EO) | 2.5% |
| Stearyl alcohol | 1% |
| Cetyl alcohol | 1% |
| Hydrogenated polyisobutene | 20% |
| Phase B | |
| Carbomer (carboxylvinyl polymer) | 0.3% |
| Triethanolamine | 0.3% |
| Water | 10% |
| Phase C | |
| Polyglycerolated dioleate | 1% |
| Ursolic acid (supplied by NIKKOL) | 0.4% |
| Water | qs 8.6% |
| Phase D | |
| Preservatives | qs |
| Water | qs 100% |

The appearance of crystalline lumps was observed after one month at room temperature and at 45° C.

French patent application 99 15612 filed Dec. 10, 1999, is incorporated herein by reference.

1. A composition comprising at least 0.02% of at least one pentacyclic triterpenic acid and at least one emulsifier, wherein said emulsifier is an optionally oxyalkylenated fatty acid monoester which is liquid at 25° C. and has an HLB greater than or equal to 12.

2. The composition according to claim 1, wherein said pentacyclic triterpenic acid is selected from the group consisting of ursolic acid and oleanolic acid.

3. The composition according to claim 1, wherein said pentacyclic triterpenic acid is at least 50% by weight in free acid form.

4. The composition according to claim 1, wherein said fatty acid monoester is formed from a fatty acid comprising from 12 to 22 carbon atoms and a polyol, and optionally oxyethylenated.

5. The composition according to claim 4, wherein said fatty acid monoester is formed from a fatty acid comprising from 12 to 22 carbon atoms and from polyglycerol.

6. The composition according to claim 4, wherein said fatty acid monoester is formed from a fatty acid comprising from 12 to 22 carbon atoms and from sorbitan, and wherein said monoester is polyoxyethylenated.

7. The composition according to claim 1, wherein the pentacyclic triterpenic acid represents from 0.05 to 5% of the total weight of the composition.

8. The composition according to claim 1, wherein the pentacyclic triterpenic acid represents from 0.4 to 2% of the total weight of the composition.

9. The composition according to claim 1, wherein the fatty acid monoester represents from 0.05 to 10% of the total weight of the composition.

10. The composition according to claim 1, wherein the fatty acid monoester represents from 1 to 5% of the total weight of the composition.

11. The composition according to claim 1, further comprising from 30 to 95% by weight of water.

12. The composition according to claim 11, comprising from 50 to 80% by weight of water.

13. A method of solubilizing a pentacyclic triterpenic acid, comprising mixing said acid with at least one optionally oxyalkylenated fatty acid monoester which is liquid at 25° C. and has an HLB greater than or equal to 12.

14. The method according to claim 13, further comprising homogenizing the mixture of acid and monoester at room temperature.

15. The method according to claim 14, further comprising introducing the homogenized mixture into an O/W or W/O emulsion at room temperature.

16. A method for the prevention or treatment of acne, of photoageing and/or of sensitive skin, comprising topically applying a composition according to claim 1 to the skin.
